(19) United States
(12) Patent Application Publication
(10) Pub. No.: US 2024/0252330 A1
(43) Pub. Date: Aug. 1, 2024

(54) EXTERNAL CRANIAL PROSTHESIS

(71) Applicant: CORNELL UNIVERSITY, Ithaca, NY (US)

(72) Inventors: Susan C. Pannullo, Essex Fells, NJ (US); Karli Thornton, Lubbock, TX (US); Stacey Kim, Bakersfield, CA (US); Shwetha Sairam, Belle Mead, NJ (US); Tyler Nicole Webb, Plano, TX (US); Joseph Miller, New Hope, PA (US); John Cheeseborough, Westbury, NY (US)

(21) Appl. No.: 18/560,820

(22) PCT Filed: May 18, 2022
(86) PCT No.: PCT/US22/29874

§ 371 Date: Nov. 14, 2023

Related U.S. Application Data
(60) Provisional application No. 63/189,821, filed on May 18, 2021.

(51) Int. Cl. A61F 2/78

(52) U.S. Cl. CPC A61F 2/7812

(57) ABSTRACT

A cranial prosthesis can be locatable adjacent to an exterior portion of a head and can include a body defining an interior surface locatable adjacent to the exterior portion of the head. The prosthesis can include a projection extending laterally outward along a first edge of the body and a slot extending inward along a second edge of the body, the slot configured to receive a projection of an adjacent prosthesis to secure the cranial prosthesis to an adjacent prosthesis.

### BACKGROUND

Decompressive craniectomy procedures involve temporary removal of a portion of a skull of a patient to relieve pressure or swelling of the patient's brain. Such swelling can be caused by a stroke, traumatic brain injury, or intracranial infection. A portion of patient's skull bone may also be removed for disease, such as tumor, infection, or injury of the skull bone itself. Often, the resected cranial portion (removed skull bone) is stored or implanted in the abdominal wall of the patient for an extended period of time while swelling of the brain reduces. In other situations, the bone is unusable and discarded. While the cranial portion is removed, the patient's brain is without its normal protection provided by the skull and extra care must be taken to avoid injuring the patient's brain until the cranial portion can be replaced or reconstructed. In some situations, the bone can never be replaced; in those situations, the patient is permanently vulnerable to injury without an external mechanism to protect the intracranial contents.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a side perspective view of a patient.

FIG. 1B illustrates a side perspective view of a patient.

FIG. 1C illustrates a side perspective view of a patient.

FIG. 2A illustrates a perspective view of a cranial prosthesis.

FIG. 2B illustrates a perspective view of a cranial prosthesis assembly.

FIG. 2C illustrates a side perspective view of a patient.

FIG. 3A illustrates an isometric front view of a cranial prosthesis.

FIG. 3B illustrates an isometric rear view of a cranial prosthesis.

FIG. 3C illustrates an isometric top view of a cranial prosthesis.

FIG. 3D illustrates an isometric side view of a cranial prosthesis.

FIG. 4 illustrates an isometric front view of a cranial prosthesis assembly.

FIG. 5A illustrates an isometric rear view of a cranial prosthesis.

FIG. 5B illustrates an isometric front view of a cranial prosthesis.

FIG. 5C illustrates an isometric side view of a cranial prosthesis.

FIG. 5D illustrates an isometric side view of a cranial prosthesis.

FIG. 6A illustrates an isometric front view of a cranial prosthesis assembly.

FIG. 6B illustrates an isometric front view of a cranial prosthesis assembly.

FIG. 7A illustrates a side perspective view of a patient and a portion of a cranial prosthesis assembly.

FIG. 7B illustrates a side perspective view of a patient and a portion of a cranial prosthesis assembly.

FIG. 8 illustrates a side perspective view of a cranium and a portion of a cranial prosthesis assembly.

### DETAILED DESCRIPTION

Following a decompressive craniectomy procedure and before a reconstruction or replacement or reconstruction procedure, a patient's brain is without its normal protection provided by the skull. Often, helmets or external prostheses are used to protect the cranial bore (or opening) that is covered only by scalp and other soft tissues. Cranial helmets and large external prostheses can be cumbersome to manipulate, uncomfortable to wear and cosmetically unappealing.

This disclosure helps to address these issues by providing a prosthesis assembly that is modular. One or more components or panels can be connected to form a prosthesis assembly that is sized and shaped to cover the cranial bore or opening without surrounding a large portion of the cranium of the patient (i.e., without surrounding more of the skull than necessary). The panels or assembly can be small enough to be secured under normal headwear such as a cap, hat, or hairpiece (wig), which can help to enhance patient comfort and appearance, and can help to increase a likelihood of patient use, and hence safety, before the reconstruction procedure can be performed.

The above discussion is intended to provide an overview of subject matter of the present patent application. It is not intended to provide an exclusive or exhaustive explanation of the invention. The description below is included to provide further information about the present patent application.

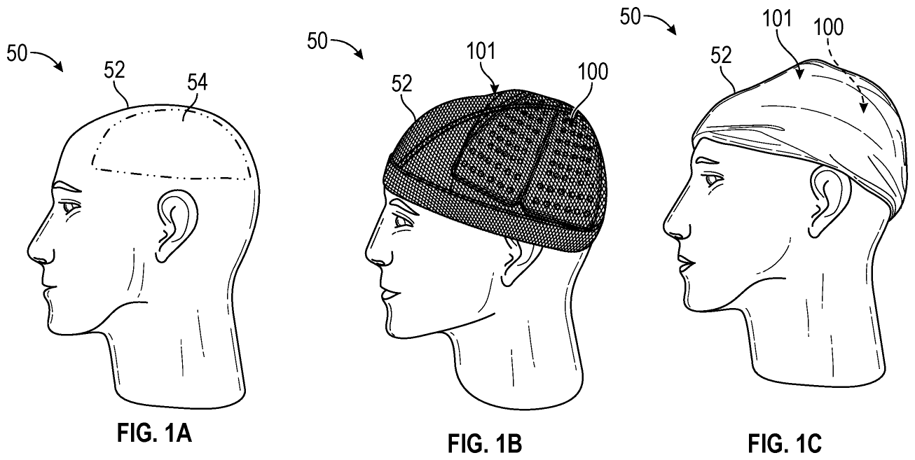

FIG. 1A illustrates a side perspective view of a patient 50. FIG. 1B illustrates a side perspective view of the patient 50. FIG. 1C illustrates a side perspective view of the patient 50. FIGS. 1A-1C are discussed together below.

FIG. 1A shows a cranium 52 of the patient 50 with a cranial bore 54 extending through the cranium or skull 52 of the patient and covered by a surface of the scalp of the patient. Such an opening can leave the patient's brain relatively unprotected from external cranial impact. Though the opening or bore 54 is shown as being on a side of the patient's 50 cranium 52, the bore 54 can be in any location. FIGS. 1B and 1C show how an implant assembly 100 can be used to cover FIG. 1B shows a prosthesis assembly 100 under a liner or stocking cap 101.

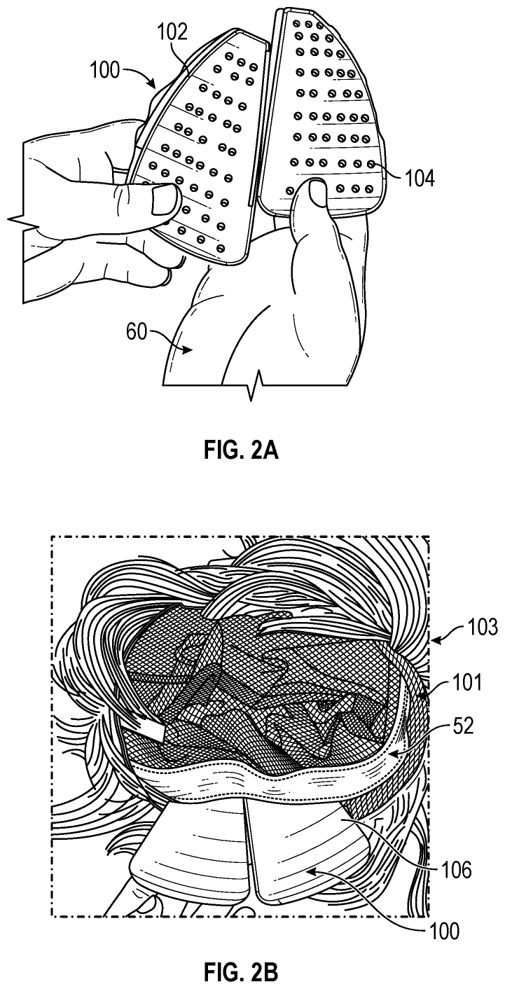

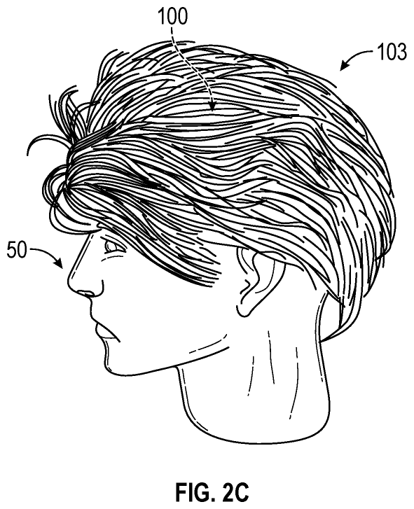

FIG. 2A illustrates a perspective view of a cranial prosthesis assembly 100. FIG. 2A shows how a user 60 (e.g., physician or patient) can secure a first prosthesis 102 to a second prosthesis 104 to form the assembly 100. A number or size of prostheses used can be selected based on the patient or use, such as based on a size or shape of the cranial bore 54 or opening in the cranium 52 of the patient 50.

FIG. 2B illustrates a perspective view showing how the cranial prosthesis assembly 100 can be assembled and inserted into a liner 101 of a hairpiece 103. For example, as shown in FIG. 2B, the assembly 100 can be positioned between the liner 101 and the hair or a second liner of the hairpiece 103 to secure the assembly 100 to the hairpiece. Optionally, the assembly 100 can be sewn (or otherwise secured) to the liner 101 or the hairpiece 103, such as using bores of the assembly 100. FIG. 2C shows how the prosthesis assembly 100 can be hidden by the hairpiece 103 following placement onto the patient's 50 head.

FIG. 2B also shows padding 106 that can be securable to an interior surface or portion of the prosthesis assembly, such as to help absorb or distribute a load applied to the portions 102 or 104 to a head (e.g., skin, scalp, hair, or cranium 52) of the patient 50 around the opening 56. The padding 106 can be made of one or more of foam, cloth, or the like.

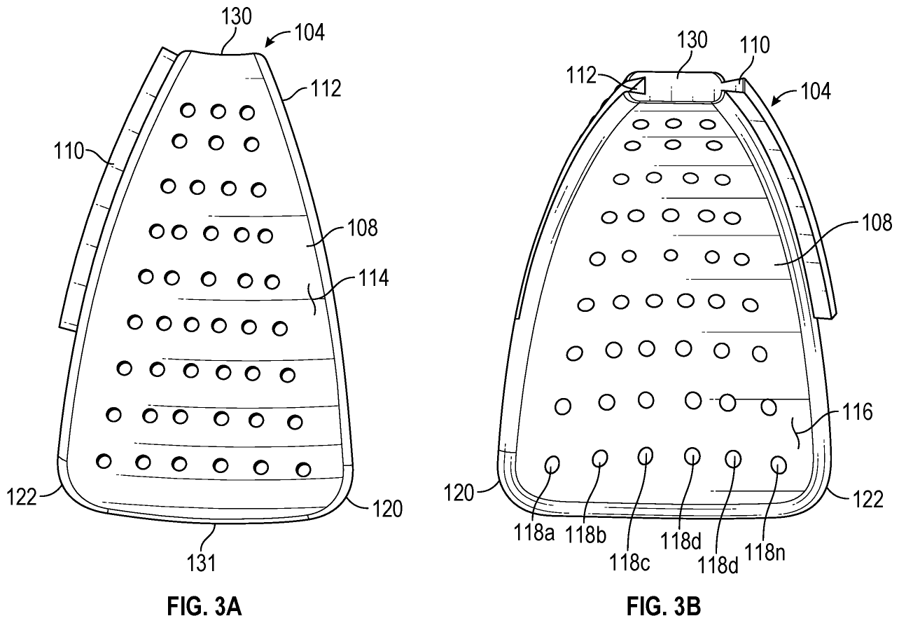

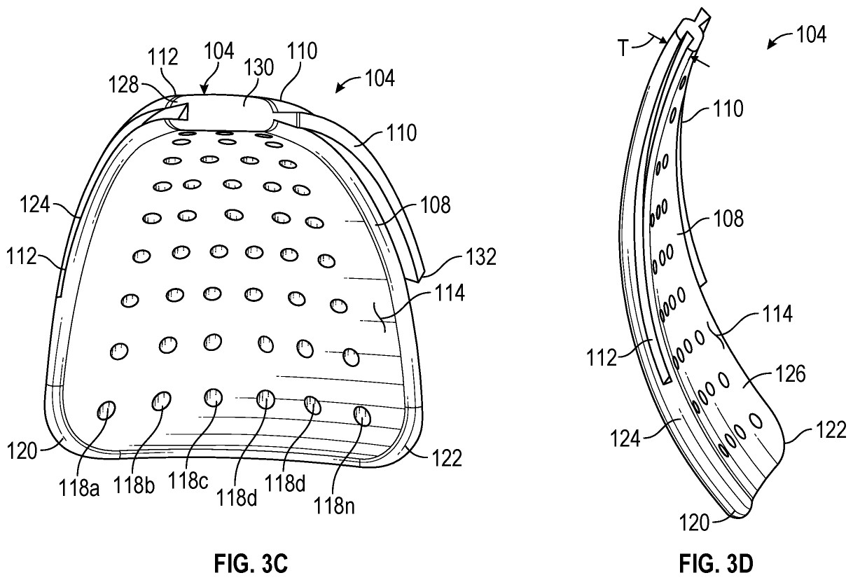

FIG. 3A illustrates an isometric front view of the cranial prosthesis 104. FIG. 3B illustrates an isometric rear view of the cranial prosthesis 104. FIG. 3C illustrates an isometric top view of the cranial prosthesis 104. FIG. 3D illustrates an isometric side view of the cranial prosthesis 104. FIGS. 3A-3D are discussed together below.

The cranial prosthesis 104 can include a body 108 that can be a rigid or semi-rigid body made of materials such as one or more of metals, plastics, foams, elastomers, ceramics, composites, combinations thereof, or the like, such as polypropylene or acrylonitrile butadiene styrene (ABS). The prostheses 104 can optionally be made of one or more of Polyetheretherketone (PEEK), polycarbonate, polyethylene terephthalate (PET), titanium, polytetrafluoroethylene (PTFE), polypropylene, or Kydex. The cranial prosthesis 104 can be manufactured through injection molding, three-dimensional printing (3D printing or additive manufacturing) or the like.

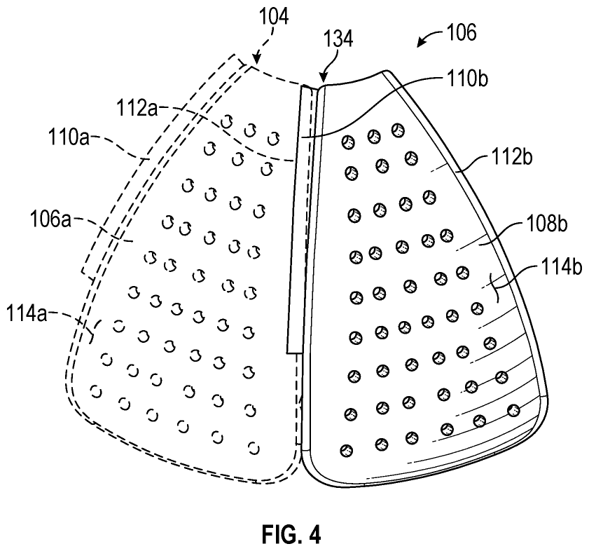

The body 108 can include a projection 110 extending from one side or edge of the body 108 and the body 108 can include a slot 112 extending into another side or edge of the body 108 opposite the projection 110. The projection 110 can have a trapezoidal cross-sectional shape or other non-symmetrical shape. The slot 112 can be shaped complimentary to the projection 110 such as to be able to receive a projection 110 of another prosthesis component or panel. As discussed below with respect to FIG. 4, the slot 112 can form a dovetail engagement with a projection when the slot 112 receives the projection therein to secure one panel or prosthesis to another through a sliding interface or engagement. The body 108 can have a thickness between 1 millimeter (mm) and 20 millimeters. Optionally, the body 108 can have a thickness between 4 and 6 mm.

The body 108 can define an exterior surface 114 that can be relatively smooth and can be (with the body 108, generally) configured to distribute a load applied to the exterior surface 114 across an interior surface 116 of the body 108. As such, the interior surface 116 can be configured to distribute the received load from the exterior surface 114 across the interior surface 116, such as to help limit transfer of load (or force, etc.) through the prosthesis assembly and to the brain of the patient, such as through the opening 54 of the cranium 52.

The body 108 can have an overall shape of a curved trapezoid from the front perspective of FIG. 3A, such as having or defining curved sides, a curved bottom portion 131, and a curved top portion 130. Optionally the top portion 130 can terminate at a point such that the body 108 can have a curved triangular shape. Curvature of the edges of the body can help to improve patient comfort. The shape of the body 108 can also be curved along the internal surface 114 and external surface 116, such as to conform to the skull or cranium of an average person. The shape can help to allow multiple prostheses 104 to be joined and to revolve around the head like a helmet, as discussed in FIG. 8 below. Furthermore, the shape can accommodate the sliding dovetail joint.

Optionally, the body 108 can be made or shaped to be patient-specific. Optionally, the assembly 100 can be provided in a variety of sizes that fit most patients to allow for a physician or patient to piece together an assembly that covers the cranial opening 56 and matches a curvature and size of the patient's cranium.

The body 108 can also define a plurality of bores 118a-118n therein that can extend therethrough. The bores 118 can be between 1 millimeter in diameter and 10 millimeters (mm) in diameter. In some examples, the bores can be between 2 mm and 5 mm, such as 3 mm. The bores 118a-118n can all be the same size or can be various sizes. The bores 118 can help to allow for air and moisture to pass through the prosthesis 104, such as when the prosthesis 104 is engaged with, covering, at least partially covering, positioned over, located over, surrounding, at least partially surrounding, or locatable adjacent to the cranium 52 of the patient. The bores 118 can also allow for the body 108 (and therefore the prosthesis 104 or assembly 100) to be sewn or otherwise secured to a piece of headwear, such as a liner, hat, hairpiece, headband, or the like. The bores 118 can be optionally arranged in a pattern on or through the body 108 to balance a maximum number of bores that provide strength of the body 108 sufficient to protect the patient's brain by distributing load around the cranial bore, helping to optimize comfort and effectiveness.

The body 108 can also define curved corners 120 and 122, which can further help to increase patient comfort and decrease palpability. Similarly, as shown in FIG. 3D, an edge 124 of the body 108 (such as the edge that defines the slot 112) can be rounded or curved. An edge 126 (opposite the edge 124) can be similarly curved or rounded.

FIG. 3C also shows an opening 128 of the slot 112 that can extend through a top portion 130 of the body 108. The opening 128 can be configured (e.g., sized or shaped) to receive a lead portion 132 of the projection such as to allow the projection 110 (such as of another prosthesis panel) to insert into the slot 112 to secure one panel to another in a sliding engagement or arrangement of the prostheses 102 and 104.

FIG. 4 illustrates an isometric front view of the cranial prosthesis assembly 100 including prostheses 102 and 104, where each of the prostheses 102 and 104 can be consistent with the prosthesis 104 of FIGS. 3A-3B. Such an assembly 100 can be made of 1, 2, 3, 4, 5, 6, 7, 8, 9, 10, or the like prostheses of the same size and shape or of different shapes or sizes.

FIG. 4 shows how the prostheses (e.g., individual panels or components) 102 and 104 can be secured to form a joint 134. The joint 134 can be formed by the projection 110b being inserted or secured within the slot 112a, as shown in FIG. 4. The joint 134 can be configured to transmit forces between the prostheses 102 and 104 to help distribute forces applied to the exterior surfaces 114a and 114b between the prostheses 102 and 104 to help transmit such forces around a cranial bore or opening (e.g., opening 56). The thickness of the joint 134 can be between 0.1 and 10 mm, but can optionally be between 2 and 3 mm.

The prostheses 102 and 104 can be assembled by sliding the projection 110b into the slot 112a (such as via the opening 128 and the portion 132) until the projection 110b is fully inserted into the slot 112a, as shown in FIG. 4. The projection 110b and slot 112a can form a dovetail engagement to help limit unintended separation of the prostheses 102 and 104, such as by pulling the prostheses 102 and 104 away from each other, such that practical separation of the prostheses 102 and 104 can be performed by sliding the projection 11b out of the slot 112a, helping to improve structural integrity of the joint 134 and the prosthesis assembly 100.

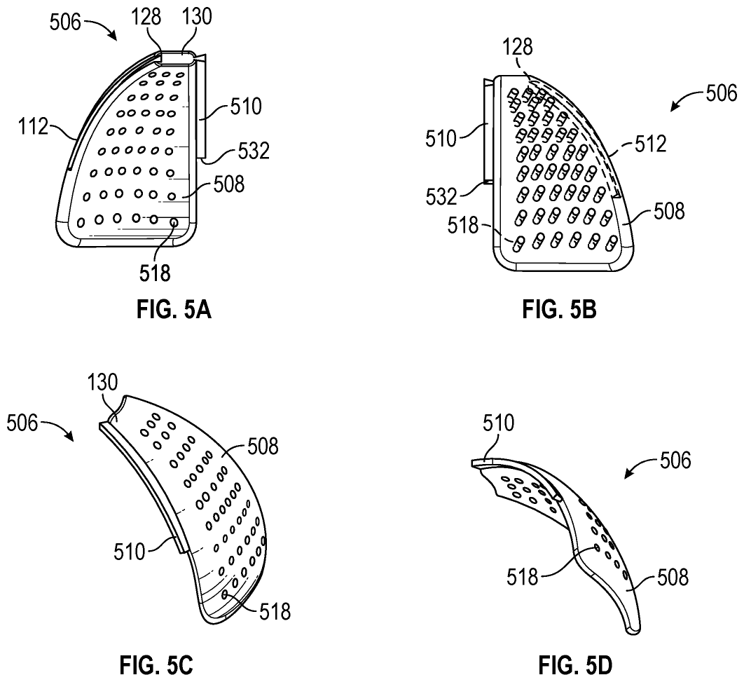

FIG. 5A illustrates an isometric rear view of a cranial prosthesis 504. FIG. 5B illustrates an isometric front view of the cranial prosthesis 504. FIG. 5C illustrates an isometric side view of the cranial prosthesis 540. FIG. 5D illustrates an isometric side view of the cranial prosthesis 504. FIGS. 5A-5D are discussed together below.

The prosthesis 504 can be similar to or the same as the prostheses 102 and 104 discussed above, such that like numerals can represent like components. FIGS. 5A-5D show additional vies and details of the prosthesis 504, such as an outer portion having a convex surface and an inner portion having a concave portion. FIG. 5B also shows that bores 518 can extend through a body 508 of the cranial prosthesis 504.

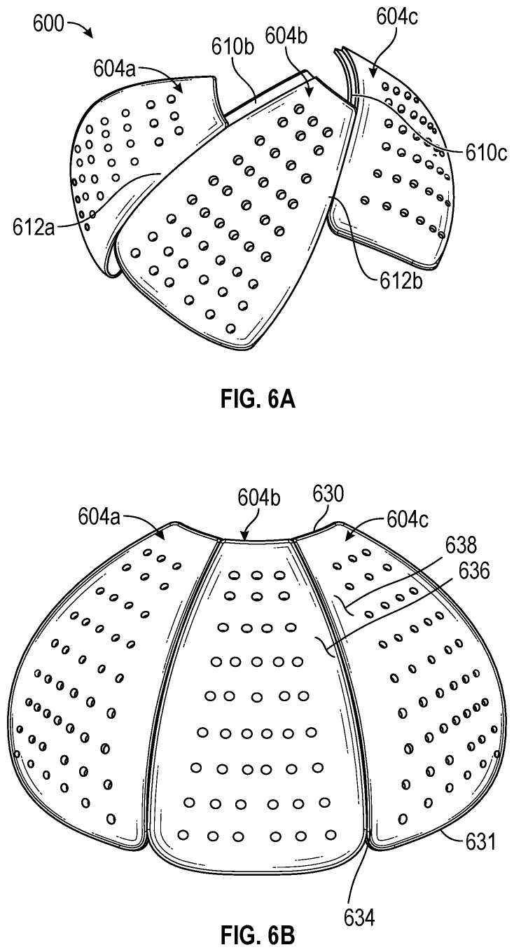

FIG. 6A illustrates an isometric front view of a cranial prosthesis assembly 600. FIG. 6B illustrates an isometric front view of the cranial prosthesis assembly 600. FIGS. 6A and 6B are discussed together below.

The cranial prosthesis assembly 600 can be similar to the assembly 100 discussed above. FIGS. 6A and 6B show how the cranial prosthesis assembly 600 can include more than two prostheses 604. For example, the cranial prosthesis assembly 600 can include a first prosthesis 604a, a second prosthesis 604b, and a third prosthesis 604c.

Each prosthesis 604 can include a body 608, a projection 610, and a slot 612. Each projection 610 can extend laterally outward along a first edge of the body 608. Each slot 612 can extend inward along a second edge of the body 608 opposite the first edge. Each projection 610 can be insertable into each slot 612 of another prosthesis 604 such that the prostheses 604 are interchangeable. Though three prostheses are shown in the cranial prosthesis assembly 600, an assembly can include 1, 2, 4, 5, 6, 7, 8, 9, 10, or the like prostheses. In some examples, as shown in FIGS. 6A and 6B, the prosthesis 604a can be provided without a projection. In this way, the prosthesis 604a can be used on an end or as an end prosthesis. By not including a projection, the prosthesis 604a can have a smooth or more smooth end surface, helping to improve patient comfort and palpability.

As shown in FIG. 6A, the projection 610b of the second prosthesis 604b can be inserted into the slot 612a of the first prosthesis 604a. The projection 610c of the third prosthesis 604c can be inserted into the slot 612b of the second prosthesis 604b. The prostheses 604 can be further manipulated until the projection 610b of the second prosthesis 604b is fully inserted into the slot 612a of the first prosthesis 604a and the projection 610c of the third prosthesis 604c is fully inserted into the slot 612b of the second prosthesis 604b, as shown in FIG. 6B. The prostheses 604 can be connected all at once or one at a time. Optionally, when connected, the prostheses 604 can be secured to each other using one or more of fasteners or adhesives. Also, the prostheses 604 can be disconnected by sliding the projections 610 out of the slots.

In the assembled configuration, shown in FIG. 6B, the cranial prosthesis assembly 600, the top portions 630 can form a first radius of curvature and the bottom portions 631 can form a second radius of curvature that is smaller than the first radius of curvature such as to allow the cranial prosthesis assembly 600 to conform to a skull or cranium.

FIGS. 6A and 6B also show that the individual prostheses 604 can be shaped to have a relatively flat surface when the prostheses 604 are assembled. For example, an outer surface 636 of the second prosthesis 604b and an outer surface 638 of the third prosthesis 604c can each be shaped such that when the second prosthesis 604b and the third prosthesis 604c are connected to form a join 634, the outer surfaces 636 and 638 align or are relatively consistent. This can help to create an overall more consistent or smooth appearance of the cranial prosthesis assembly 600 when under a piece of headwear.

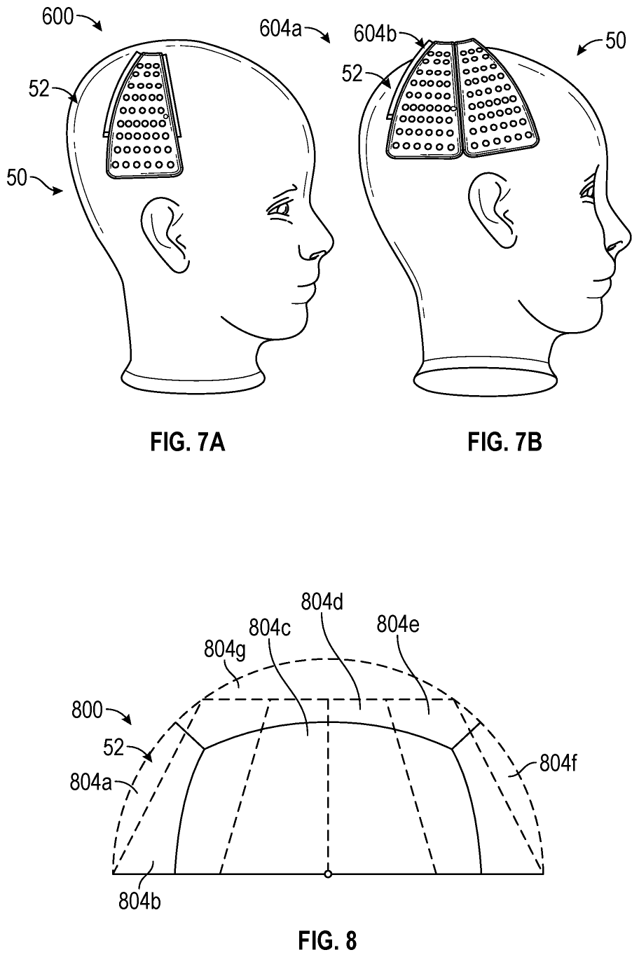

FIG. 7A illustrates a side perspective view of a patient 50 and a portion of the cranial prosthesis assembly 600. FIG. 7B illustrates a side perspective view of the patient 50 and a portion of the cranial prosthesis assembly 600. FIGS. 7A and 7B are discussed together below.

The cranial prosthesis assembly 600 of FIGS. 7A and 7B can be consistent with the cranial prosthesis assembly 600 discussed above with respect to FIGS. 6A and 6B; FIGS. 7A and 7B show how the prostheses 604 can be connected to each other, as required or desired, to cover the opening 54 of the cranium 52 of the patient 50 and to distribute forces applied to the cranial prosthesis assembly 600. As discussed with reference to FIG. 8 below, additional prostheses can be added to the cranial prosthesis assembly 600, as required or desired.

FIG. 8 illustrates a side perspective view of a cranium 52 and a portion of a cranial prosthesis assembly 800. The cranial prosthesis assembly 800 can be similar to the assembly 100 or the cranial prosthesis assembly 600 discussed above. FIG. 8 shows how the cranial prosthesis assembly 800 can include several prostheses 804 that can be connected or assembled to surround the cranium 52, either entirely or partially.

For example, a prosthesis 804a can be connected to a prosthesis 804b. The prosthesis 804b can be connected to a prosthesis 804c, which can be connected to a prosthesis 804d. The prosthesis 804d can be connected to a prosthesis 804e, which can be connected to a prosthesis 804f. A top prosthesis 804g can be connected to one or more of the prostheses 804a-804f. The top prosthesis 804g can also be connected to one or more prostheses on a lateral side of the cranium 52 opposite the prostheses 804a-804f. In this way, the prostheses 804 can be assembled to create a complete cranial support or covering. Optionally, one or more of the prostheses 804a-804g can be excluded to form a partial cranial support or covering.

### NOTES AND EXAMPLES

The following non-limiting examples, detail certain aspects of the present subject matter to solve the challenges and provide the benefits discussed herein, among others.

Example 1 is a cranial prosthesis locatable adjacent to an exterior portion of a head, the prosthesis comprising: a body defining an interior surface locatable adjacent to the exterior portion of the head; a projection extending laterally outward along a first edge of the body; and a slot extending inward along a second edge of the body, the slot configured to receive a projection of an adjacent prosthesis to secure the cranial prosthesis to an adjacent prosthesis.

In Example 2, the subject matter of Example 1 optionally includes a pad secured to the interior surface and locatable adjacent to the exterior portion of the head.

In Example 3, the subject matter of any one or more of Examples 1-2 optionally include wherein the body defines a plurality of bores extending therethrough.

In Example 4, the subject matter of any one or more of Examples 1-3 optionally include wherein the body is configured to distribute a load applied to an exterior surface of the body across the interior surface of the body.

In Example 5, the subject matter of any one or more of Examples 1˜4 optionally include wherein the body has a shape of a curved trapezoid.

In Example 6, the subject matter of any one or more of Examples 1-5 optionally include wherein the body includes corners that are rounded.

In Example 7, the subject matter of any one or more of Examples 1-6 optionally include wherein the slot and the projection of the adjacent prosthesis are configured to form a dovetail interface.

Example 8 is a cranial prosthesis assembly locatable adjacent to an exterior portion of a head, the assembly comprising: a first prosthesis comprising: a first body defining a first interior surface locatable adjacent to the exterior portion of the head; a first projection extending laterally outward along a first edge of the first body; and a first slot extending inward along a second edge of the first body; and a second prosthesis comprising: a second body defining a second interior surface locatable adjacent to the exterior portion of the head; and a second slot extending inward along a second edge of the second body, the second slot configured to receive the first projection to secure the second prosthesis to the first prosthesis.

In Example 9, the subject matter of Example 8 optionally includes a pad secured to the first interior surface or the second interior surface and locatable adjacent to the exterior portion of the head.

In Example 10, the subject matter of any one or more of Examples 8-9 optionally include wherein the first body defines a first plurality of bores extending therethrough and wherein the second body defines a second plurality of bores extending therethrough.

In Example 11, the subject matter of any one or more of Examples 8-10 optionally include wherein the first body and the second body are configured to distribute a load applied to exterior surfaces of the first body and the second body across the interior surface of the first body and the interior surface of the second body.

In Example 12, the subject matter of any one or more of Examples 8-11 optionally include wherein the first slot and the second projection form a dovetail interface.

In Example 13, the subject matter of any one or more of Examples 8-12 optionally include a third prosthesis comprising: a third body defining a third interior surface locatable adjacent to the exterior portion of the head; and a third projection extending laterally outward along a first edge of the third body, the third projection insertable into the first slot or the second slot to secure the third prosthesis to the first prosthesis or the second prosthesis; and third a third slot extending inward along a second edge of the second body, the third slot configured to receive the first projection to secure the first prosthesis to the third prosthesis.

Example 14 is a cranial prosthesis assembly locatable adjacent to an exterior portion of a head, the assembly comprising: a plurality of prostheses, each prosthesis comprising: a body defining an interior surface locatable adjacent to the exterior portion of the head; a projection extending laterally outward along a first edge of the body; and a slot extending inward along a second edge of the body, the slot configured to receive a projection of an adjacent prosthesis of the plurality of prostheses to secure the cranial prosthesis to an adjacent prosthesis.

In Example 15, the subject matter of Example 14 optionally includes a pad secured to the interior surface and locatable adjacent to the exterior portion of the head.

In Example 16, the subject matter of any one or more of Examples 14-15 optionally include wherein the body defines a plurality of bores extending therethrough.

In Example 17, the subject matter of any one or more of Examples 14-16 optionally include wherein the body is configured to distribute a load applied to an exterior surface of the body across the interior surface of the body.

In Example 18, the subject matter of any one or more of Examples 14-17 optionally include wherein the body has a shape of a curved trapezoid.

In Example 19, the subject matter of any one or more of Examples 14-18 optionally include wherein the body includes corners that are rounded.

In Example 20, the subject matter of any one or more of Examples 14-19 optionally include wherein the slot and the projection of the adjacent prosthesis are configured to form a dovetail interface.

Example 21 is a prosthesis assembly including any of the features discussed herein.

Example 22 is a prosthesis including any of the features discussed herein.

Example 23 is a method of installing a prosthesis assembly including any of the steps discussed herein.

In Example 24, the apparatuses or method of any one or any combination of Examples 1-23 can optionally be configured such that all elements or options recited are available to use or select from.

Example 25 is a cranial prosthesis locatable adjacent to an exterior portion of a head, the prosthesis comprising a first body defining an interior surface locatable adjacent to the exterior portion of the head and further defining one of a first projection extending laterally outward along a first edge of the first body or a first slot extending inward along a second edge of the first body, wherein the one of the first projection extending laterally outward along the first edge of the first body or the first slot extending inward along the second edge of the first body is configured to matingly engage, respectively, a similarly configured second slot or second projection of a second body.

Example 26 is a cranial prosthesis according to Example 25, wherein the cranial prosthesis further comprises the second body, the second body defining an interior surface locatable adjacent to the exterior portion of the head and further defining one of the second projection, extending laterally outward along a first edge of the second body or a second slot extending inward along a second edge of the second body, wherein the one of the second projection extending laterally outward along the first edge of the second body or the second slot extending inward along the second edge of the second body is configured to matingly engage, respectively, the first slot or first projection of the first body.

Example 27 is a cranial prosthesis according to Example 26, wherein the first body defines both a first projection extending laterally outward along a first edge of the first body and a first slot extending inward along a second edge of the first body.

Example 28 is a cranial prosthesis according to Example 27, wherein the second body defines at least the second projection, extending laterally outward along a first edge of the second body.

Example 29 is a cranial prosthesis according to any one of Example 26 to Example 28, wherein the first body and the second body have a similar shape, a similar size, or both a similar shape and a similar size.

Example 30 is a cranial prosthesis according to any one of Example 26 to Example 28, wherein the first body and the second body have a dissimilar shape, a dissimilar size, or both a dissimilar shape and a dissimilar size.

Example 31 includes a cranial prosthesis according to any one of Examples 25-30, the cranial prosthesis including any combination of elements from any one or more of Examples 1-22, in any combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B.” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A cranial prosthesis locatable adjacent to an exterior portion of a head, the prosthesis comprising:
a body defining an interior surface locatable adjacent to the exterior portion of the head;
a projection extending laterally outward along a first edge of the body; and
a slot extending inward along a second edge of the body, the slot configured to receive a projection of an adjacent prosthesis to secure the cranial prosthesis to an adjacent prosthesis.

2. The prosthesis of claim 1, further comprising:
a pad secured to the interior surface and locatable adjacent to the exterior portion of the head.

3. The prosthesis of claim 1, wherein the body defines a plurality of bores extending therethrough.

4. The prosthesis of claim 1, wherein the body is configured to distribute a load applied to an exterior surface of the body across the interior surface of the body.

5. The prosthesis of claim 1, wherein the body has a shape of a curved trapezoid.

6. The prosthesis of claim 1, wherein the body includes corners that are rounded.

7. The prosthesis of claim 1, wherein the slot and the projection of the adjacent prosthesis are configured to form a dovetail interface.

8. A cranial prosthesis assembly locatable adjacent to an exterior portion of a head, the assembly comprising:
a first prosthesis comprising:
a first body defining a first interior surface locatable adjacent to the exterior portion of the head;
a first projection extending laterally outward along a first edge of the first body; and
a first slot extending inward along a second edge of the first body; and
a second prosthesis comprising:
a second body defining a second interior surface locatable adjacent to the exterior portion of the head; and
a second slot extending inward along a second edge of the second body, the second slot configured to receive the first projection to secure the second prosthesis to the first prosthesis.

9. The assembly of claim 8, further comprising:
a pad secured to the first interior surface or the second interior surface and locatable adjacent to the exterior portion of the head.

10. The assembly of claim 8, wherein the first body defines a first plurality of bores extending therethrough and wherein the second body defines a second plurality of bores extending therethrough.

11. The assembly of claim 8, wherein the first body and the second body are configured to distribute a load applied to exterior surfaces of the first body and the second body across the interior surface of the first body and the interior surface of the second body.

12. The assembly of claim 8, wherein the first slot and the second projection form a dovetail interface.

13. The assembly of claim 8, further comprising:
a third prosthesis comprising:
a third body defining a third interior surface locatable adjacent to the exterior portion of the head; and
a third projection extending laterally outward along a first edge of the third body, the third projection insertable into the first slot or the second slot to secure the third prosthesis to the first prosthesis or the second prosthesis; and third a third slot extending inward along a second edge of the second body, the third slot configured to receive the first projection to secure the first prosthesis to the third prosthesis.

14. A cranial prosthesis assembly locatable adjacent to an exterior portion of a head, the assembly comprising:
a plurality of prostheses, each prosthesis comprising:
a body defining an interior surface locatable adjacent to the exterior portion of the head;
a projection extending laterally outward along a first edge of the body; and
a slot extending inward along a second edge of the body, the slot configured to receive a projection of an adjacent prosthesis of the plurality of prostheses to secure the cranial prosthesis to an adjacent prosthesis.

15. The prosthesis of claim 14, further comprising:
a pad secured to the interior surface and locatable adjacent to the exterior portion of the head.

16. The prosthesis of claim 14, wherein the body defines a plurality of bores extending therethrough.

17. The prosthesis of claim 14, wherein the body is configured to distribute a load applied to an exterior surface of the body across the interior surface of the body.

18. The prosthesis of claim 14, wherein the body has a shape of a curved trapezoid.

19. The prosthesis of claim 14, wherein the body includes corners that are rounded.

20. The prosthesis of claim 14, wherein the slot and the projection of the adjacent prosthesis are configured to form a dovetail interface.
